(19) United States
(12) Patent Application Publication
(10) Pub. No.: US 2025/0352716 A1
(43) Pub. Date: Nov. 20, 2025

(54) ENEMA DELIVERY DEVICE FOR PEOPLE WITH IMPAIRED MOBILITY

(71) Applicant: Enable Independence, LLC, Kamas, UT (US)

(72) Inventors: Matthew R. Carter, Kamas, UT (US); Noah Gostout, Rochester, MN (US)

(21) Appl. No.: 18/666,621

(22) Filed: May 16, 2024

(51) Int. Cl. A61M 3/02

(52) U.S. Cl. CPC A61M 3/0262; A61M 3/0202; A61M 2205/13; A61M 2210/1067

(57) ABSTRACT

An enema delivery device includes an arm assembly, a body assembly, and a stopper mechanism. The arm assembly includes a compression region, a first grip region that includes a first coupling mechanism, and a first bend region that has a pivot axis that passes through it. The body assembly includes a holding region configured to hold a cartridge, and a second grip region that includes a second coupling mechanism. The body assembly also includes a second bend region that couples the second grip region and the holding region such that the holding region is canted from the second grip region. The second bend region couples to the first bend region such that both the arm assembly and the body assembly pivot about the pivot axis. The stopper mechanism is coupled to the holding region and controls compression of the cartridge by the compression region.

### FIELD OF THE INVENTION

This disclosure relates generally to enema delivery, and more specifically to enema delivery devices for people with impaired mobility.

### BACKGROUND

One in four adults in the United States, approximately sixty-one million individuals, are living with disability. Disabilities can affect an individual's cognitive ability, mobility, ability to live independently, or care for themselves. Mobility impairments comprise the largest category of the disabled population, approximately 14%. In comparison with the 4% of Americans who are unable to independently perform activities of daily living, such as: dressing, bathing, bowel, and bladder management. Therefore, there are a limited number of resources being dedicated to solving and optimizing independence barriers for those who have difficulty performing activities of daily living independently.

Bowel management, in particular, can be a problem for people with impaired mobility that limits their ability to control the fine motor skills in their hands (e.g., grasping a small object, creating pressure to eject a substance from a container, etc.). Conventional techniques to manage constipation use methods that are based on an individual having fine motor control of their hands and are largely not able to be used by these people, thereby inhibiting their ability to manage their self-care independently and potentially live independently.

### SUMMARY

Embodiments of enema delivery devices (“delivery devices”) are described herein. The ability to create enough pressure on a receptacle with specific hand function may be the single limiting factor to an individual living independently. The delivery devices described herein mitigate this issue. The delivery devices described may be operated using gross motor movements of users (v. fine motor movements). Embodiments of the delivery devices may be used with different types of enema cartridges (e.g., cartridge). In some embodiments, the delivery devices may be mechanically triggered to expel fluid (e.g., liquid medication) from an enema cartridge. In alternate embodiments, a delivery device may be remotely and/or electronically triggered.

In some aspects, the techniques described herein relate to a device including: an arm assembly that includes: a compression region, a first grip region that includes a first coupling mechanism, and a first bend region that couples the first grip region and the compression region such that the compression region is canted from the first grip region, and a pivot axis passes through the first bend region; a body assembly that includes: a holding region configured to hold a cartridge, a second grip region that includes a second coupling mechanism that is configured to couple to the first coupling mechanism, and a second bend region that couples the second grip region and the holding region such that the holding region is canted from the second grip region, and the second bend region is coupled to the first bend region such that both the arm assembly and the body assembly pivot about the pivot axis; and a stopper mechanism coupled to the holding region and configured to control compression of the cartridge by the compression region.

In some aspects, the techniques described herein relate to a method including: loading a device with a cartridge, wherein the device includes: an arm assembly that includes: a compression region, a first grip region that includes a first coupling mechanism, and a first bend region that couples the first grip region and the compression region such that the compression region is canted from the first grip region, and a pivot axis passes through the first bend region, a body assembly that includes: a holding region configured to hold the cartridge, a second grip region that includes a second coupling mechanism that is configured to couple to the first coupling mechanism, and a second bend region that couples the second grip region and the holding region such that the holding region is canted from the second grip region, and the second bend region is coupled to the first bend region such that both the arm assembly and the body assembly pivot about the pivot axis, and a stopper mechanism coupled to the holding region and configured to control compression of the cartridge by the compression region; causing the first grip region and the second grip region to come together such that the first coupling mechanism couples to the second coupling mechanism and a stopper mechanism is in a first position to prevent the compression region from rotating about the pivot axis creating tension in the body assembly and the arm assembly; and triggering a release mechanism of the device to move the stopper mechanism to a second position that allows the compression region to rotate about the pivot axis toward the holding region to release the tension and compress the cartridge to cause the cartridge to expel its contents.

### BRIEF DESCRIPTION OF THE DRAWINGS

FIG. 1 illustrates an example delivery device, in accordance with one or more embodiments

FIG. 2A illustrates the delivery device of FIG. 1 in a loading state.

FIG. 2B illustrates the delivery device of FIG. 1 in a loaded state.

FIG. 2C illustrates the delivery device of FIG. 1 in a triggered state.

FIG. 2D illustrates an alternate view of the delivery device 100 of FIG. 2C.

FIG. 3 illustrates is an exploded view of some of the delivery device of FIG. 1.

FIG. 4 illustrates a delivery device and corresponding cartridge, in accordance with one or more embodiments.

FIG. 5 is a flowchart illustrating a process for operating a delivery device, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

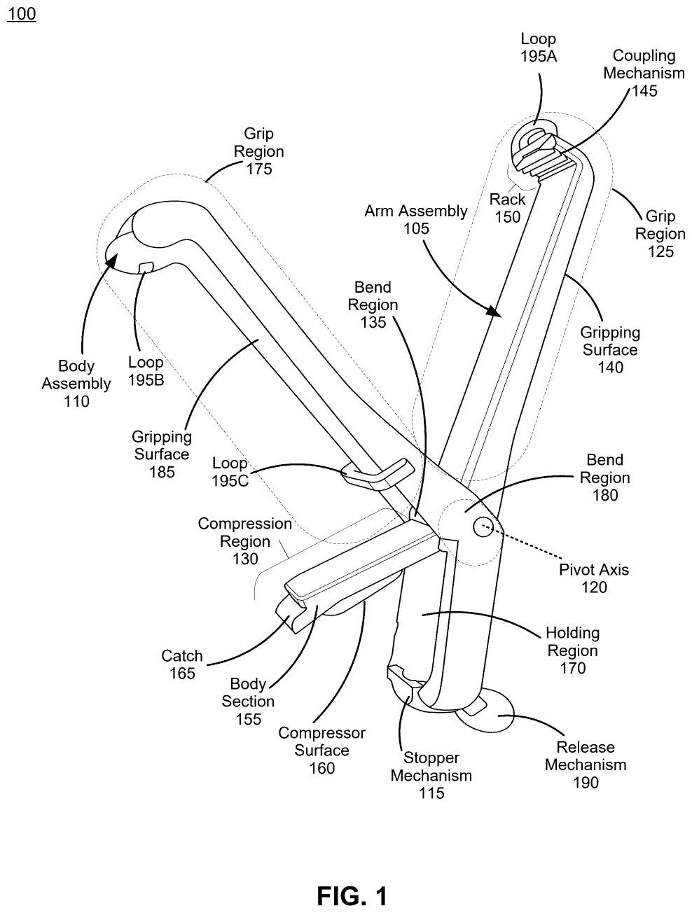

FIG. 1 illustrates an example delivery device 100, in accordance with one or more embodiments. The delivery device 100 is for delivering fluid (e.g., liquid medication) from an enema cartridge into a rectum of a user. The delivery device 100 includes an arm assembly 105, a body assembly 110, and a stopper mechanism 115. Alternative embodiments may include more, fewer, or different components from those illustrated in FIG. 1, and the functionality of each component may be divided between the components differently from the description below.

An enema cartridge is a cartridge that includes a fluid (e.g., to treat constipation and/or some other medical condition). In some embodiments, the enema cartridge includes a reservoir that holds the fluid and a nozzle. Compression of a portion (e.g., the reservoir) of the enema cartridge past a threshold pressure causes the fluid to pass from the reservoir into the nozzle which expels the fluid from the enema cartridge. In some embodiments, the enema cartridge may also include a collar between the reservoir and the nozzle. The collar may be positioned on the enema cartridge to help control how far the nozzle is inserted into the rectum. In some embodiments the nozzle may be blocked with a removable cap, tab, etc. to prevent accidental expulsion of the fluid. The enema cartridge may sometimes be referred to as a “cartridge.” Note, in some embodiments, the enema cartridge does not include a nozzle, and the nozzle may be part of the delivery system 100. In these embodiments, the reservoir would be punctured and expelled through the nozzle of the delivery system 100.

The arm assembly 105 may function as a lever that rotates about a pivot axis 120. The arm assembly 105 is composed of a grip region 125, a compression region 130, and a bend region 135. In some embodiments, the arm assembly 105 is monolithic such that the grip region 125, the compression region 130, and the bend region 135 are all are formed from a same piece of material. The material may be, e.g., a polymer (e.g., Acrylonitrile Butadiene Styrene), Polyvinyl chloride, Polyethylene terephthalate (PET), Polyethylene terephthalate glycol (PETG), Polypropylene, HDPE, LDPE, fiber reinforced injection compounds, metal, or some combination thereof. For example, the arm assembly 105 may be fabricated out of a single material using injection molding, three-dimensional (“3D”) printing, etc. In alternate embodiments, some or all of the components of the arm assembly 105 may be individually fabricated (e.g., 3D printed) and then coupled together to form the arm assembly 105. For example, the grip region 125 and the bend region 135 may be a single monolithic component that is coupled to the compression region 130 which is a separate monolithic component.

The grip region 125 is a portion of the arm assembly 105 that the user may interact with to rotate the arm assembly 105 about the pivot axis 120. The grip region 125 includes a gripping surface 140 and a coupling mechanism 145. The gripping surface 140 is a surface of the arm assembly 105 designed to be in contact with a portion (e.g., fingers, hand, palm, wrist, thumb) of an appendage of the user as they manipulate the arm assembly 105. In some embodiments, the gripping surface 140 may be shaped to fit to a particular surface (e.g., such that the delivery device 100 is stable while the user lowers themself onto it), to be more ergonomic, to be shaped to fit a specific mobility impairment of the user, or some combination thereof. For example, it may be shaped to conform to a surface of a toilet seat. In another example, the delivery device 100 is configured to fit on the toilet seat and be down out of the way and then the delivery device 100 could be brought up into the rectum. This may be helpful for people with impaired mobility where it would be extremely hard to lower correctly on the device without causing trauma. In some embodiments, the gripping surface 140 be coated with anti-slip material.

The coupling mechanism 145 is configured to couple to a coupling mechanism of the body assembly 110. In the illustrated embodiments, the coupling mechanism 145 includes a rack 150 with a first set of teeth (e.g., the sawtooth shaped projections from the rack 150).

The compression region 130 is configured to compress the enema cartridge once the delivery device 100 has been triggered. The compression region 130 includes a body section 155, a compressor surface 160, and a catch 165. The body section 155 couples the compression region 130 to the bend region 135, and provides structural support for the compressor surface 160 and the catch 165. The compressor surface 160 extrudes from the body section 155 towards a holding region 170. The holding region is a location in which a portion of the enema cartridge may be placed. The catch 165 that extrudes from an end of the body section 155 such the catch 165 may come into contact with the stopper mechanism 115 as the arm assembly 105 and/or the body assembly 110 are rotated about the pivot axis 120.

The bend region 135 couples the grip region 125 to the compression region 130. The coupling is such that the compression region 130 is canted from the grip region 125, and the pivot axis 120 passes through the bend region 135.

The body assembly 110 may function as a lever that rotates about the pivot axis 120. The body assembly 110 is composed of the holding region 170, a grip region 175, and a bend region 180. In some embodiments, the body assembly 110 is monolithic such that the holding region, the grip region 175, and the bend region 180 are all are formed from a same piece of material. In alternate embodiments, some or all of the components of the body assembly 110 may be individually fabricated (e.g., 3D printed) and then coupled together to form the body assembly 110. For example, the grip region 175 and the bend region 180 may be a single monolithic component that is coupled to the holding region 170. The body assembly 110 may be composed of the same materials as the arm assembly 105. The body assembly 110 may also be fabricated using the same or similar techniques as those described above with regard to fabrication of the arm assembly 105.

The holding region 170 is configured to hold an enema cartridge. The holding region includes at least three sides. The stopper mechanism 115 and the compressor surface 160 work together with the holding region 170 to hold the enema cartridge within the holding region 170.

The grip region 175 is a portion of the arm assembly 105 that the user may interact with to rotate the body assembly 110 about the pivot axis 120. The grip region 175 includes a gripping surface 185 and a coupling mechanism (not visible in FIG. 1). The gripping surface 185 is a surface of the body assembly 110 designed to be in contact with a portion (e.g., fingers, hand, palm, wrist, thumb) of an appendage of the user as they manipulate the body assembly 110. In some embodiments, the gripping surface 140 may be shaped to fit to a portion of the appendage (e.g., shaped to fit fingers, thumb, or wrist). In some embodiments, the gripping surface 185 be coated with anti-slip material.

The coupling mechanism is configured to couple to the coupling mechanism 145 of the arm assembly 105. The coupling mechanism may include a rack with a second set of teeth. The second set of teeth are configured to interface with the first set of teeth to ratchet the grip region 125 and the grip region 175 together and cause the compression region 130 to move toward the stopper mechanism 115. In some embodiments, the rack of the coupling mechanism on the body assembly 110 and the rack 150 are shaped such that they are in contact and can be ratcheted together while the stopper mechanism 115 is positioned to prevent the compression region 130 from rotating about the pivot axis 120 such that tension in the body assembly 110 and the arm assembly 105 is created. The rack and the rack 150 may also be shaped such that after the stopper mechanism 115 is positioned (e.g., triggered) to allow the compression region 130 to rotate about the pivot axis 120 toward the holding region 170 to release the tension that the two racks separate such that the grip region 125 and the grip region 175 can rotate away from each other about the pivot axis 120.

The bend region 180 couples the grip region 175 to the holding region 170. The coupling is such that the holding region 170 is canted from the grip region 175, and the bend region 180 is coupled to the bend region 135 such that both the arm assembly 105 and the body assembly 110 pivot about the pivot axis 120. An interior surface of the bend region 180 faces an exterior surface of the bend region 135, and the compression region 130 and the grip region 125 are on opposite sides of the grip region 175.

Note that the arm assembly 105 is narrower than the body assembly 110, such that the compression region 130 can fit within the holding region 170. Similarly, as the arm assembly 105 is narrower than the body assembly 110, portions of the grip region 125 may fit within a portion of the grip region 175.

The stopper mechanism 115 is coupled to the holding region 170 and configured to control compression of the enema cartridge by the compression region 130. The stopper mechanism includes an aperture (not visible in FIG. 1). The aperture is sized to allow a nozzle of the enema cartridge to extrude from it while the enema cartridge is within the holding region 170. The stopper mechanism 115 includes a release mechanism 190.

In some embodiments, the stopper mechanism 115 is monolithic is formed from a same piece of material. In alternate embodiments, some or all of the components of the stopper mechanism 115 may be individually fabricated (e.g., 3D printed) and then coupled together to form the stopper mechanism 115. The stopper mechanism 115 may be composed of the same materials as the arm assembly 105. The stopper mechanism 115 may also be fabricated using the same or similar techniques as those described above with regard to fabrication of the arm assembly 105. In some embodiments, the release mechanism 190 may include one or more portions that are made out of a soft material (e.g., foam, silicon, etc.) to make it more comfortable for the user to trigger the delivery device 100.

The release mechanism 190 is configured to move the stopper mechanism 115 through a range of motion from a first position to a second position. As shown in FIG. 1, the stopper mechanism 115 in positioned in the first position. In the first position the stopper mechanism 115 is configured to prevent the compression region 130 from applying more than a holding pressure on an enema cartridge within the holding region 170. The holding pressure is a pressure that is high enough to hold an enema cartridge within the holding chamber, but not above the threshold pressure that would cause the enema cartridge to expel the fluid. The release mechanism 190 may be used to rotate the stopper mechanism 115 to the second position. In the second position, the stopper mechanism 115 is configured to allow the compression region 130 to apply pressure in excess of the holding pressure on the enema cartridge. In the illustrated embodiments, the release mechanism 190 is a tab that can be moved by the user of the delivery device 100. In some embodiments, it may be moved by a hand of the user, soft tissue of the buttocks (e.g., lowering themselves onto the delivery device 100), etc.

In some embodiments (not shown) the delivery device 100 includes one or more proximity sensors. The one proximity sensors may be placed near the aperture of the stopper mechanism 115. The one or more proximity sensors may be configured to detect when a nozzle of the enema cartridge has been inserted into a lower bowel of a patient by way of a rectum of the patient, and generate one or more trigger signals. Likewise in some embodiments the delivery device 100 may receive a trigger signal from a remote input device. A remote triggering device is a device that is separate from the delivery device 100 and generates trigger signal(s). A triggering device may be, e.g., a pedal that is communicatively coupled to the delivery device 100 and that the user may trigger the delivery device 100, an app on a mobile computing device, a switch (e.g., a button on a wheelchair), etc.

The delivery device 100 may include a controller (not shown) that triggers the delivery device 100 responsive to receiving one or more trigger signals. The controller may be configured to control a position of the stopper mechanism 115 to allow compression of the cartridge based in part on received trigger signal(s) (e.g., from the one or more proximity sensors, a remote input device, etc.). For example, the release mechanism may be spring loaded to be in the second position, and can be held in the first position with an electro-magnet that is controlled by the controller. The controller may, e.g., receive one or more signals (e.g., a remote input, one or more proximity sensors, etc.) and electronically trigger the release mechanism 190 by deactivating the electro-magnet which would cause the stopper mechanism 115 to move to the second position.

In some embodiments, the delivery device 100 may also include one or more actuators that can control movement of the body assembly 110, the arm assembly 105, the stopper mechanism 115, or some combination thereof. The one or more actuators may be controlled via the controller. In this manner, the user may provide a trigger to the controller to place the delivery device 100 in various states of operation as described below. For example, the one or more actuators may cause the body assembly 110 and/or the arm assembly 105 to rotate about the pivot axis 120.

In some embodiments, the delivery device 100 may include one or more feedback devices to alert the user that the delivery device 100 has triggered. A feedback means may be, e.g., an audible click made by the delivery device 100 as it triggers, a haptic feedback device, a speaker, a light emitting diode, etc. In some embodiments, a controller may be communicatively coupled to a mobile device and instruct the mobile device to provide feedback that corresponds to the various states. For example, the controller may instruct the mobile device to vibrate, emit a sound, flash its display, etc., once the delivery device has triggered. In some embodiments, the type of feedback provided is based on which state the delivery device 100 is currently in (e.g., unloaded, triggered, etc.).

In some embodiments, the delivery device 100 includes one or loops to which a strap may be attached. For example, in the illustrated embodiment, the arm assembly 105 includes a loop 195A, and the body assembly 110 includes a loop 195B and a loop 195C. In other embodiments, there may no loops, or some other number of loops. The one or more loops may be located in different positions on the arm assembly 105 and/or the body assembly 110.

In some embodiments, portions of the delivery device 100 may be configured to couple to a reaching instrument. The reaching instrument may be useful to a user with limited mobility as it can provide additional reach for the user to position the delivery device 100.

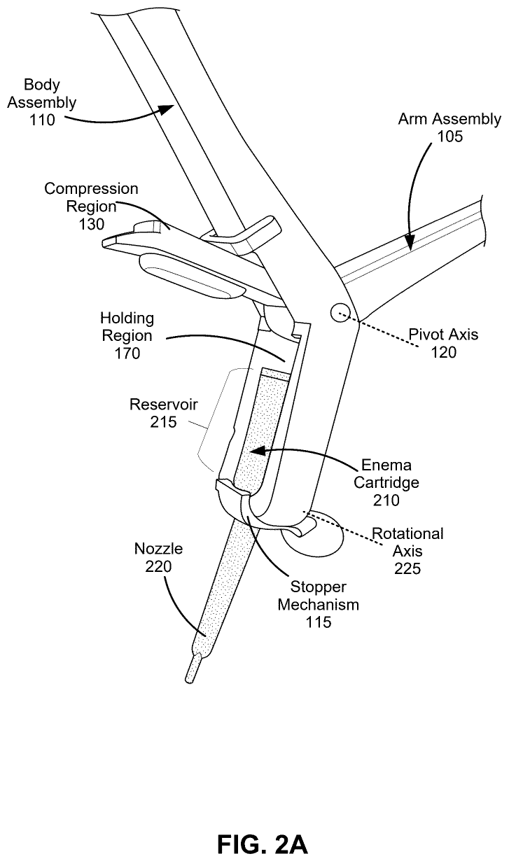

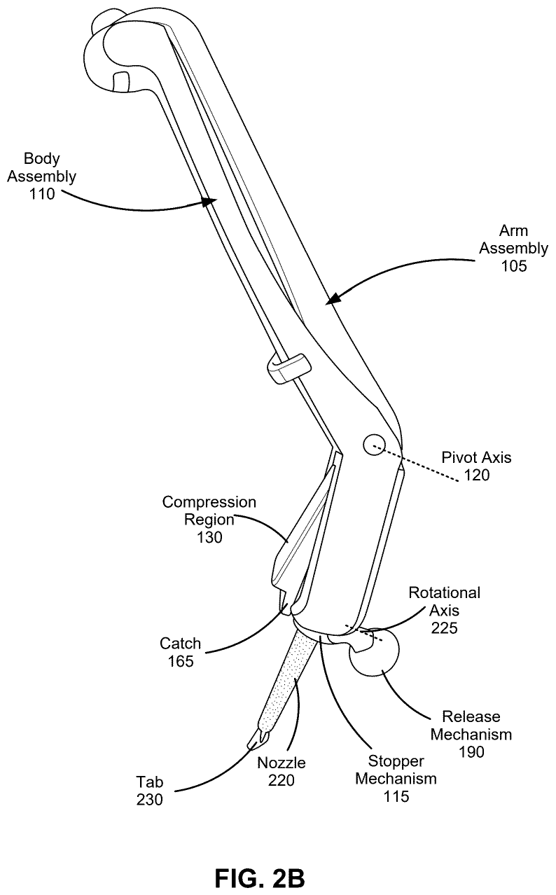

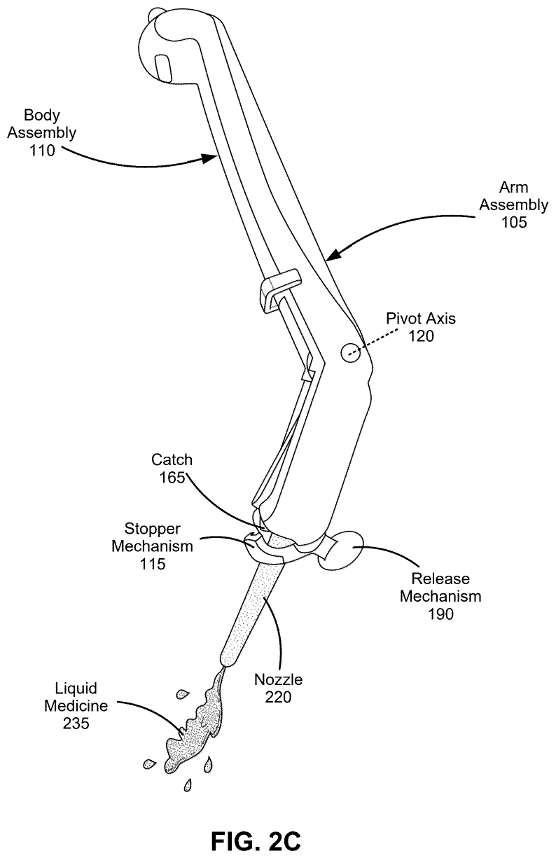

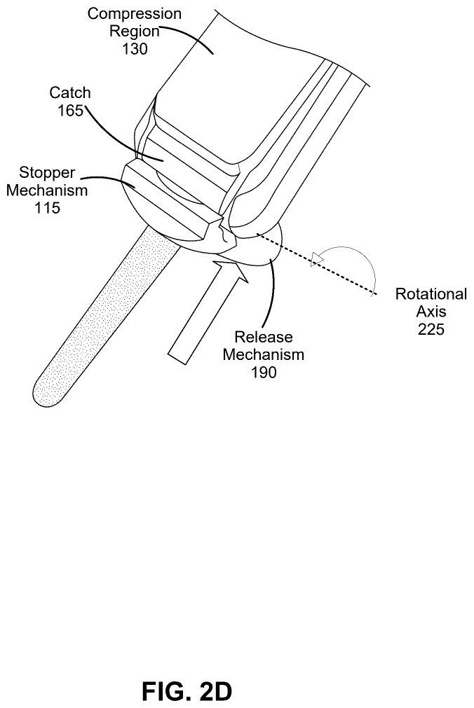

The delivery device 100 may be placed in various states for operation that are described in detail below. The states include: an unloaded state, a loading state, a loaded state, and a triggered state. The delivery device 100 illustrated in FIG. 1 is in the unloaded state. In the unloaded state, the delivery device 100 is not holding an enema cartridge. The remaining states are described in detail below with respect to FIGS. 2A-D.

FIG. 2A illustrates the delivery device 100 of FIG. 1 in a loading state. In the loading state, the delivery device 100 is holding an enema cartridge 210, but the coupling mechanisms of the arm assembly 105 and the body assembly 110 are not coupled together.

The enema cartridge 210 may be placed such that a reservoir 215 of the enema cartridge 210 is within the holding region 170 and a nozzle 220 is placed through the aperture (not visible in FIG. 2A) in the stopper mechanism 115.

The stopper mechanism 115 in positioned in the first position. In the first position the stopper mechanism 115 is configured to prevent the compression region 130 from applying more than a holding pressure on an enema cartridge within the holding region 170. The stopper mechanism 115 is configured to rotate about a rotational axis 225 that passes through the stopper mechanism 115 and is parallel to the pivot axis 120. The stopper mechanism 115 may be held in the first position via a holding means. The holding means may be, e.g., small protrusions (e.g., bumps) on the stopper mechanism that snap into corresponding indentations in the holding region 170 or vice versa. In some embodiments, the holding means may be spring based. In some embodiments the holding means is magnet based. For example, the stopper mechanism 115 may include a first magnet, and the holding region 170 may include a second magnet, and the two magnets are positioned to hold the stopper mechanism in the first position. One advantage of the holding means being magnet based is that the one or more magnets may be encapsulated in the material and therefore reduces surfaces for biofilm build up.

After the enema cartridge has been placed in the holding region 170, the arm assembly 105 and/or the body assembly 110 are rotated about the pivot axis 120 such that a distance between their respective grip regions becomes smaller and the compression region 130 comes in contact with the stopper mechanism 115. Once the distance between the two grip regions is small enough, the coupling mechanisms can couple (e.g., ratchet) together and place the delivery device 100 in a loaded state.

FIG. 2B illustrates the delivery device 100 of FIG. 1 in a loaded state. In the loaded state, the coupling mechanisms of the arm assembly 105 and the body assembly 110 are coupled together such that tension in the body assembly 110 and the arm assembly 105 is created. In the loaded state, the two coupling mechanisms are coupled together and the stopper mechanism 115 is positioned to prevent the compression region 130 from rotating about the pivot axis 120 such that tension in the body assembly and the arm assembly is created. As the grip assemblies 127, 125 of the body assembly 110 and the arm assembly 105 are further compressed together, the coupling mechanisms may further ratchet together increasing the tension. The tension is in effect potential energy. And once the delivery device 100 is triggered, a portion of the potential energy is converted to compressive force that is applied to the reservoir 215 of the enema cartridge 210.

In the illustrated embodiment, the enema cartridge 210 includes a tab 230 on the nozzle 220 to prevent accidently expulsion of the fluid. In some embodiments, prior to triggering the delivery device 100, the tab 230 should be removed. To trigger the delivery device 100, the release mechanism 190 is rotated slightly such that the stopper mechanism 115 rotates away from the catch 165 of the compression region 130.

FIG. 2C illustrates the delivery device 100 of FIG. 1 in a triggered state. In FIG. 2C, the delivery device 100 has been triggered, such that the stopper mechanism 115 has been moved to a position (e.g., the second position) where the stopper mechanism 115 no longer blocks the compression region 130 allowing the tension in the arm assembly 105 and/or the body assembly 110 to release, resulting in the compression region 130 suddenly applying pressure in excess of the holding pressure on the enema cartridge 210. The sudden application of pressure pushes fluid 235 from the reservoir 215 through the nozzle 220 which is then expelled into the local area.

While not visible in FIG. 2C, the release in tension also causes the coupling mechanisms to decouple. Thereby allowing, the arm assembly 105 and the body assembly 110 to once again freely rotate about the pivot axis 120. This facilitates being able to easily put the move the grip regions 125, 175 away from each other, to allow the compression region 130 to move away from the holding region 170. The used enema cartridge 210 can be removed to place the delivery device 100 back in an unloaded state as shown and described above with regard to FIG. 1.

Note that a user may operate the delivery device 100 using gross motor movements like wrist extension and elbow flexion to facilitate this fluid ejection. In contrast, conventional enema delivery devices are operated using fine motor movements (e.g., pinching of fingers or similar fine motor grip, etc.). The ability to be operated using gross motor movement, can be the single factor that allows an individual with a disability to manage their self-care independently and therefore potentially live independently. Note that constipation can cause numerous health issues which can ultimately cause sepsis and death. The delivery device 100 may allow an individual to no longer require a caregiver, which can be burdensome on one's personal finances as well as insurance companies bottom lines. Independence also creates increased privacy with bowel management. Accordingly, the delivery device 100 may allow for effective neurogenic bowel management in people with impaired functional mobility. The delivery device 100 will provide consistent force production and therefore ejection volume, all thing equal, because it is a repeatable process, it could be a means of delivering a more precise volume of medicine. As compared to the variability of a user squeezing the cartridge with their fingers. Additionally, the delivery device 100 may be operated by a second person remotely (e.g., in another room), thereby providing greater privacy to the patient receiving the enema.

FIG. 2D illustrates an alternate view of the delivery device 100 of FIG. 2C. As noted above, in the triggered state, the stopper mechanism 115 has been moved to a position (e.g., the second position) where the stopper mechanism 115 no longer blocks the compression region 130. In the illustrated embodiment, a force applied to the release mechanism 190 causes the stopper mechanism 115 to rotate about the rotational axis 225. The rotation moves the stopper mechanism 115 into a position where it no longer supports the catch 165 thereby allowing for the sudden release in tension in the arm assembly 105 and/or the body assembly 110 as described above with regard to FIG. 2C.

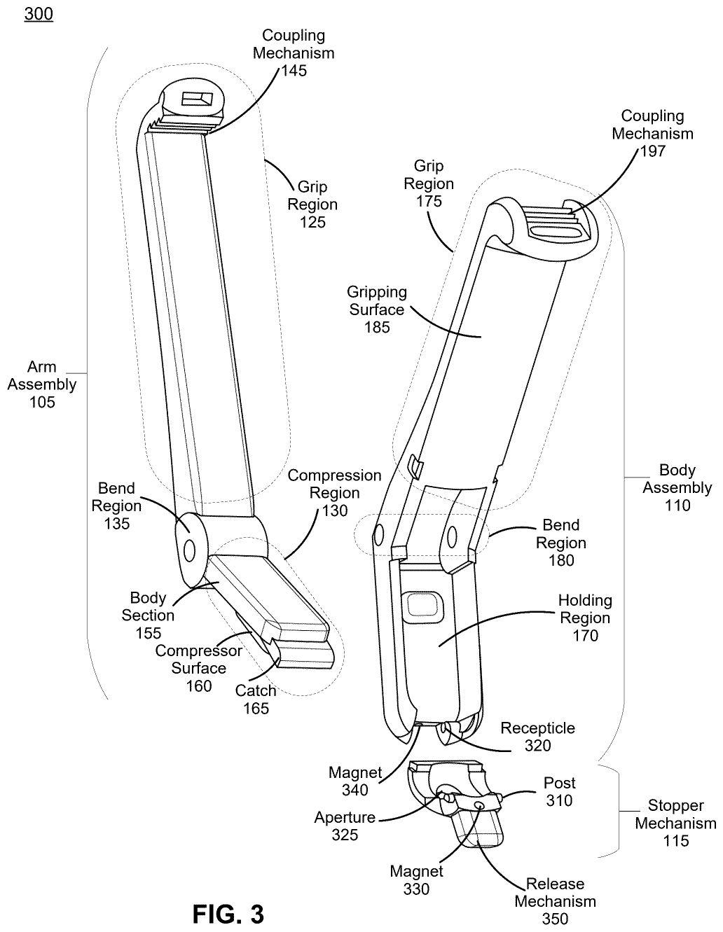

FIG. 3 illustrates is an exploded view 300 of some of the delivery device 100 of FIG. 1. The delivery device is an embodiment of the delivery device 100. The exploded view 300 shows the arm assembly 105, the body assembly 110, and the release mechanism 190 in isolation. The arm assembly 105 and the body assembly 110 include respective coupling mechanisms 145, 197. In the illustrated embodiment, each of the coupling mechanism 145, 197 includes a respective rack and associated teeth.

As noted above the arm assembly 105 and the body assembly 110 couple together at the bend regions 138, 180. In some embodiments, the there is a pin (not shown) that passes through the bend region 138 and the bend region 180. The pivot axis 120 as shown above with regard to FIG. 1 may run through a center axis of the pin. In other embodiments, the bend regions 138, 180 couple together in some other manner. For example, the bend region 135 may include posts that couple to corresponding receptacles in the bend region 180 along the pivot axis 120, such that when coupled together the arm assembly 105 and/or the body assembly 110 can rotate about the pivot axis 120.

In the illustrated example, the stopper mechanism 115 may couple to the body assembly 110 using posts (e.g., post 310) on the stopper mechanism 115 that couple to corresponding receptacles (e.g., receptacle 320) of the body assembly 110. The posts and receptacles, when coupled together, are positioned along the rotational axis 225.

The stopper mechanism 115 includes an aperture 325. The aperture 325 is sized to allow a nozzle of an enema cartridge to extrude from it while the enema cartridge is within the holding region 170.

In the illustrated embodiment, the holding means of the stopper mechanism 115 is magnet based. For example, the stopper mechanism 115 includes a magnet 330, and the holding region 170 include a magnet 340, and the two magnets are positioned such that in the assembled delivery device 100 the magnets 330, 340 act to hold the stopper mechanism in the first position. A user may use a release mechanism 350 to adjust a position of the stopper mechanism 115 (e.g., to rotate about the rotational axis 225). The release mechanism 350 is an embodiment of the release mechanism 190 that is shaped differently.

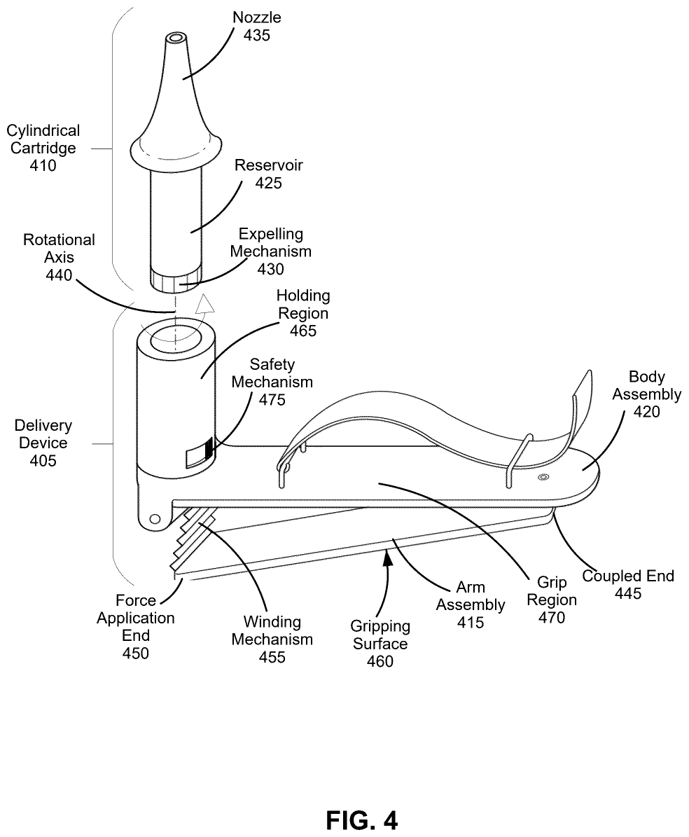

FIG. 4 illustrates a delivery device 405 and corresponding cartridge 410, in accordance with one or more embodiments. The delivery device 405 is for delivering fluid from the cartridge 410 into a rectum of a user. The delivery device 405 includes an arm assembly 415 and a body assembly 420. Alternative embodiments may include more, fewer, or different components from those illustrated in FIG. 4, and the functionality of each component may be divided between the components differently from the description below.

The cartridge 410 is a cartridge that includes a fluid (e.g., to treat constipation and/or some other medical condition). The cartridge 410 includes a reservoir 425, an expelling mechanism 430, and a nozzle 435. Note that in the illustrated embodiment, the cartridge 410 is cylindrical. In other embodiments (not shown), the cartridge 410 may have some other shape (e.g., rectangular, pouch, etc.), and the delivery device 405 is modified to use this other shape. The reservoir 425 is configured to hold the fluid. The expelling mechanism 430 forces the fluid from the reservoir 425 into the nozzle 435 from which it is expelled into a local area. The expelling mechanism 430 includes a geared platform that rotates about a rotational axis 440. The geared platform translates rotational force to translation force along the rotational axis toward the nozzle 435. For example, as the expelling mechanism 430 spins about the rotational axis 440 it causes the geared platform to move into the reservoir toward the nozzle 435. The geared platform may be geared such that a small change in rotational position of the expelling mechanism 430 results in a relatively large change in translation of the geared platform into the reservoir 425. In the illustrated embodiment, the cartridge 410 includes a depth limiter that flairs out between the reservoir and the nozzle. The depth limiter functions as the collar described above and helps control how far the nozzle is inserted into the rectum. In some embodiments the nozzle 435 may be blocked with a removable cap, tab, etc. to prevent accidental expulsion of the fluid.

The arm assembly 415 provides a linear input force to the body assembly 420. The arm assembly 415 includes a winding mechanism 455 and a gripping surface 460. In some embodiments, the arm assembly 415 is monolithic such that the gripping surface 460 and the winding mechanism 455 are all are formed from a same piece of material. In alternate embodiments, some or all of the components of the body assembly 420 may be individually fabricated (e.g., 3D printed) and then coupled together to form the arm assembly 415. The arm assembly 415 may be composed of the same materials as the arm assembly 105 as described above with regard to FIG. 1. The arm assembly 415 may also be fabricated using the same or similar techniques as those described above with regard to fabrication of the arm assembly 105.

The arm assembly includes a coupled end 445 and a force application end 450 that is opposite the coupled end 445. The coupled end 445 of the arm assembly 415 is coupled to the body assembly 420 such that an adjustable angle between the body assembly 420 and the arm assembly 415 is formed. The winding mechanism 455 is located at the force application end 450. The winding mechanism 455 is configured to wind one or more gears within the body assembly 420 as the force application end 450 moves closer to the body assembly 420.

The gripping surface 460 is a surface of the arm assembly 105 designed to be in contact with a means of manipulating the arm assembly 415. In some embodiments, gripping surface 460 is shaped to conform to a surface (e.g., a toilet seat) to be more ergonomic, to be shaped to fit a specific mobility impairment of the user, or some combination thereof. In alternate embodiments, the gripping surface 460 is shaped to fit a portion (e.g., fingers, hand, palm, wrist, thumb) of an appendage of the user. In some embodiments, the gripping surface 140 be coated with anti-slip material.

The body assembly 420 provides leverage for a user bring the body assembly 420 and the arm assembly 415 closer together. The body assembly 420 is composed of a holding region 465 and a grip region 470. In some embodiments, the body assembly 420 is monolithic such that the holding region 465 and the grip region 470 are all are formed from a same piece of material. In alternate embodiments, some or all of the components of the body assembly 420 may be individually fabricated (e.g., 3D printed) and then coupled together to form the body assembly 420. For example, the grip region 470 and the holding region 465 may be separate monolithic components that are coupled together. The body assembly 420 may be composed of the same materials as the body assembly 110 as described above with regard to FIG. 1. The body assembly 420 may also be fabricated using the same or similar techniques as those described above with regard to fabrication of the body assembly 110.

The holding region 465 is configured to hold the cartridge 410. The holding region 465 includes a cylindrical opening that is sized to fit at least a portion (e.g., the expelling mechanism 430 and the reservoir 425) of the cartridge 410. The holding region 465 includes an advancement mechanism (not visible in FIG. 4) and a safety mechanism 475.

The advancement mechanism is wound using the winding mechanism 455. For example, the advancement mechanism may be wound by pressing the arm assembly 415 such that the winding mechanism 455 moves into the advancement mechanism. The advancement mechanism may include one or more winding gears, a spring, one or more cartridge gears, and a release. The one or more input gears are rotated using the winding mechanism 455, an in turn they wind up the spring to achieve a wound state for the delivery device 405. In a wound state, the spring is wound and holding potential energy. For example, the one or more gears may be structured such that they reach a lock point after a threshold amount of rotation that winds the spring to a threshold level of potential energy.

The one or more cartridge gears are configured to rotate the expelling mechanism 430 about the rotational axis 440. The release may function to release the potential energy stored by the spring into rotation of the one or more cartridge gears, and thereby rotate the expelling mechanism 430 to expel the liquid mechanism from the cartridge 410.

In some embodiments, the release is mechanical in nature. The advancement mechanism may be configured to trigger the release based on pressure from the cartridge 410. For example, as the nozzle 435 is placed into the rectum, once the depth limiter is reached back pressure on the advancement mechanism increases greatly. The increase in pressure may be used to trigger the release. In other embodiments, the release may be electronically and/or remotely controlled. For example, the delivery device 405 may include one or more proximity sensors. The one or more proximity sensors may be configured to detect when the nozzle 435 of the cartridge 410 has been inserted into a lower bowel of a patient by way of a rectum of the patient, and generate one or more trigger signals. Likewise in some embodiments the delivery device 405 may receive a trigger signal from a remote input device. A triggering device may be, e.g., a pedal that is communicatively coupled to the delivery device 100 and that the user may trigger the delivery device 100, an app on a mobile computing device, a switch (e.g., a button on a wheelchair), etc. The delivery device 100 may include a controller that triggers the delivery device 405 responsive to receiving one or more trigger signals. The controller may be configured to trigger the delivery device 405 in part on received trigger signal(s) (e.g., from the one or more proximity sensors, a remote input device, etc.).

An example use case for the delivery device 405 is described as follows. The delivery device 405 is placed in a wound state. For example, the advancement mechanism may be wound by pressing the arm assembly 415 such that winding mechanism 455 moves into the advancement mechanism one or more times until a lock position is reached. The user loads the delivery device 405 with the cartridge 410 by inserting it into the holding region 465. The user may disengage the safety mechanism 475. The nozzle 435 begins insertion into a rectum of the user (or some other patient). The release of the advancement mechanism is triggered to release the fluid. For example, the release may be triggered based on an increase in pressure at the cartridge 410 that occurs once the depth limiter is reached during insertion.

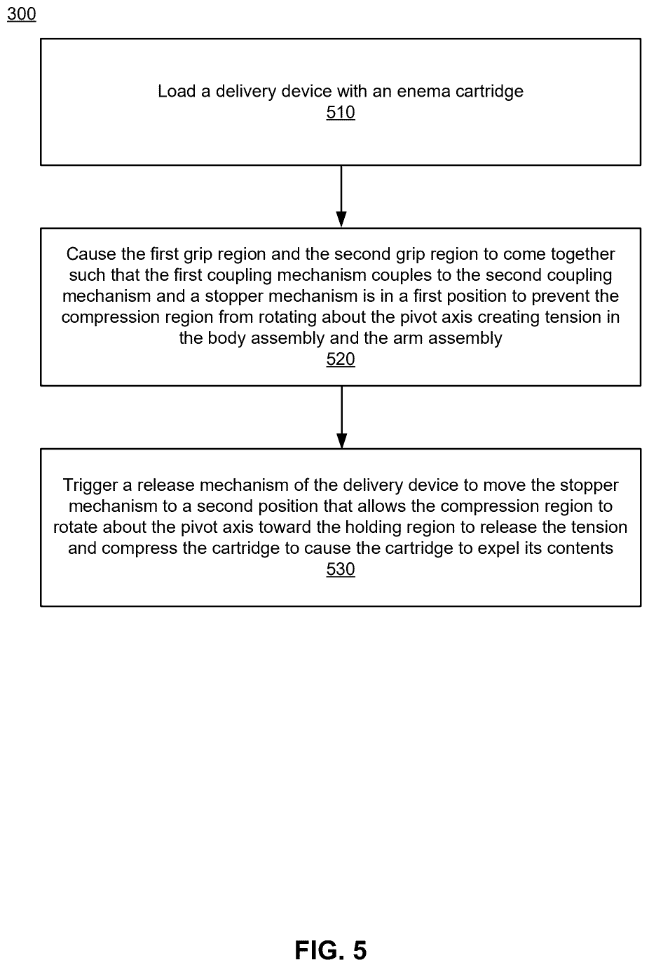

FIG. 5 is a flowchart illustrating a process 500 for operating a delivery device, in accordance with one or more embodiments. The process shown in FIG. 5 may be performed by a user operating the delivery device 100. Embodiments may include different and/or additional steps, or perform the steps in different orders.

The user loads 510 the delivery device 100 with an enema cartridge. The delivery device 100 comprises: an arm assembly, a body assembly, and a stopper mechanism. The arm assembly includes a compression region, a first grip region that includes a first coupling mechanism, and a first bend region. The first bend region couples the first grip region to the compression region such that the compression region is canted from the first grip region, and a pivot axis passes through the first bend region. The body assembly includes a holding region, a second grip region, and a second bend region. The holding region is configured to hold the enema cartridge. The second grip region includes a second coupling mechanism that is configured to couple to the first coupling mechanism. The second bend region couples the second grip region and the holding region such that the holding region is canted from the second grip region. The second bend region is coupled to the first bend region such that both the arm assembly and the body assembly pivot about the pivot axis. The stopper mechanism is coupled to the holding region and is configured to control compression of the cartridge by the compression region

The user causes 520 the first grip region and the second grip region to come together such that the first coupling mechanism couples to the second coupling mechanism and a stopper mechanism is in a first position to prevent the compression region from rotating about the pivot axis creating tension in the body assembly and the arm assembly. This puts the delivery device 100 in a loaded state. For example, the user may place the stopper mechanism in the first position, and squeeze the first grip region and the second grip region such that they come together and the first coupling mechanism couples to the second coupling mechanism. As the first coupling mechanism and the second coupling mechanism ratchet together, tension in the body assembly and the arm assembly increases. Also, the coupling of the first coupling mechanism and the second coupling mechanism is such that once a particular level of ratcheting occurs—the user may stop squeezing the first grip region and the second grip region together and the coupling mechanisms can maintain the level of ratcheting.

The user triggers 530 a release mechanism of the delivery device 100 to move the stopper mechanism to a second position that allows the compression region to rotate about the pivot axis toward the holding region to release the tension and compress the cartridge to cause the cartridge to expel its contents. In some embodiments, the release mechanism is a mechanical release (e.g., as shown in in FIG. 1) that is triggered by the user moving it. In some embodiments, the release mechanism may be triggered via a remote trigger (e.g., pedal that is coupled to the delivery device). In some embodiments, the release mechanism may be electronic.

For example, a signal from a proximity sensor on the delivery device that would cause a controller to electronically trigger the release mechanism.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A device comprising:
an arm assembly that includes:
a compression region,
a first grip region that includes a first coupling mechanism, and
a first bend region that couples the first grip region and the compression region such that the compression region is canted from the first grip region, and a pivot axis passes through the first bend region;
a body assembly that includes:
a holding region configured to hold a cartridge,
a second grip region that includes a second coupling mechanism that is configured to couple to the first coupling mechanism, and
a second bend region that couples the second grip region and the holding region such that the holding region is canted from the second grip region, and the second bend region is coupled to the first bend region such that both the arm assembly and the body assembly pivot about the pivot axis; and
a stopper mechanism coupled to the holding region and configured to control compression of the cartridge by the compression region.

2. The device of claim 1, wherein the stopper mechanism includes an aperture that a nozzle of the cartridge is configured to extrude from, and contents of the cartridge are expelled from the nozzle of the cartridge subject to compression of the cartridge by the compression region.

3. The device of claim 1, wherein the device is in a loaded state when the first coupling mechanism is coupled to the second coupling mechanism and the stopper mechanism is positioned to prevent the compression region from rotating about the pivot axis such that tension in the body assembly and the arm assembly is created.

4. The device of claim 3, wherein the device is in a triggered state when the stopper mechanism is positioned to allow the compression region to rotate about the pivot axis toward the holding region to release the tension in the body assembly and the arm assembly.

5. The device of claim 1, wherein the stopper mechanism includes:
a release mechanism configured to move the stopper mechanism from a first position to a second position, wherein in the first position the stopper mechanism is configured to prevent the compression region from applying more than a holding pressure on the cartridge, and in the second position the stopper mechanism is configured to allow the compression region to apply pressure in excess of the holding pressure on the cartridge.

6. The device of claim 5, wherein the release mechanism is a tab that is moved by a user of the device.

7. The device of claim 5, wherein the stopper mechanism is configured to rotate about a rotational axis that passes through the stopper mechanism and is parallel to the pivot axis.

8. The device of claim 7, wherein the stopper mechanism includes a first post and a second post that are positioned along the rotational axis, and the first post and the second post are configured to couple to corresponding receptacles in the holding region.

9. The device of claim 5, wherein the stopper mechanism includes a first magnet, and the body assembly holds a second magnet that are positioned to hold the stopper mechanism in the first position.

10. The device of claim 1, wherein the arm assembly is narrower than the body assembly, such that the compression region fits within the holding region.

11. The device of claim 10, wherein an interior surface of the second bend region faces an exterior surface of the first bend region, and the compression region and the first grip region are on opposite sides of the second grip region.

12. The device of claim 1, wherein the first coupling mechanism includes a first rack with a first set of teeth, and the second coupling mechanism includes a second rack with a second set of teeth configured to interface with the first set of teeth to ratchet the first grip region and the second grip region together to and cause the compression region to move toward the stopper mechanism.

13. The device of claim 12, wherein the first rack and the second rack are shaped such that: (1) they are in contact and can be ratcheted together while the stopper mechanism is positioned to prevent the compression region from rotating about the pivot axis such that tension in the body assembly and the arm assembly is created, and (2) after the stopper mechanism is positioned to allow the compression region to rotate about the pivot axis toward the holding region to release the tension the first rack and the second rack separate such that the first grip region and the second grip region can rotate away from each other about the pivot axis.

14. The device of claim 1, wherein the arm assembly is monolithic.

15. The device of claim 1, wherein the body assembly is monolithic.

16. The device of claim 1, wherein the stopper mechanism is monolithic.

17. The device of claim 1, wherein the compression region includes:
a body section;
a compressor surface that extrudes from the body section towards the holding region; and
a catch that extrudes from an end of the body section such that when the device is in a loaded state the catch is in contact with the stopper mechanism.

18. The device of claim 1, further comprising:
a proximity sensor that is configured to detect when a nozzle of the cartridge has been inserted into a lower bowel of a patient by way of a rectum of the patient; and
a controller configured to control a position of the stopper mechanism to allow compression of the cartridge based in part on a signal from the proximity sensor.

19. The device of claim 1, wherein the stopper mechanism is electronically controlled by a user of the device.

20. A method comprising:
loading a device with a cartridge, wherein the device comprises:
an arm assembly that includes:
a compression region,
a first grip region that includes a first coupling mechanism, and
a first bend region that couples the first grip region and the compression region such that the compression region is canted from the first grip region, and a pivot axis passes through the first bend region,
a body assembly that includes:
a holding region configured to hold the cartridge,
a second grip region that includes a second coupling mechanism that is configured to couple to the first coupling mechanism, and
a second bend region that couples the second grip region and the holding region such that the holding region is canted from the second grip region, and the second bend region is coupled to the first bend region such that both the arm assembly and the body assembly pivot about the pivot axis, and
a stopper mechanism coupled to the holding region and configured to control compression of the cartridge by the compression region;
causing the first grip region and the second grip region to come together such that the first coupling mechanism couples to the second coupling mechanism and a stopper mechanism is in a first position to prevent the compression region from rotating about the pivot axis creating tension in the body assembly and the arm assembly; and
triggering a release mechanism of the device to move the stopper mechanism to a second position that allows the compression region to rotate about the pivot axis toward the holding region to release the tension and compress the cartridge to cause the cartridge to expel its contents.
